(19) United States
(12) Patent Application Publication
(10) Pub. No.: US 2024/0398808 A1
(43) Pub. Date: Dec. 5, 2024

(54) DIHYDROFOLATE FOR HYPOGLYCEMIC ACTIVITY

(71) Applicant: COUNCIL OF SCIENTIFIC AND INDUSTRIAL RESEARCH an india registered boby incorporated under, New Delhi (IN)

(72) Inventors: Vikas Singh Chauhan, Karnataka (IN); Bijesh Puthusseri, West Bengal (IN); Ajana Pathikkal, Karnataka (IN); Ulaganathan Mabalirajan, West Bengal (IN); Atmaja Karmakar, West Bengal (IN); Divya Peethambaran, Karnataka (IN)

(21) Appl. No.: 18/326,450

(22) Filed: May 31, 2023

(51) Int. Cl. A61K 31/519; A23L 33/15; A61P 3/10

(52) U.S. Cl. CPC A61K 31/519; A23L 33/15; A61P 3/10; A61K 9/0053

(57) ABSTRACT

The present invention relates to the oral nutraceutical agent dihydrofolate (C₁₉H₂₁N₇O₆) for the management of hyperglycemia. These inventions have been intended to improve glucose tolerance and glycemic control in diabetic patients of any type, without causing any significant increase in weight, or risk of hypoglycemia. The present invention provides a novel approach to the treatment and prevention of type 2 diabetes and offers a significant improvement over currently available treatments. The invention also encompasses methods for reducing the incidence and severity of disease states associated with chronic insulin dosing, preserving or protecting beta-cell function, preventing beta-cell death or dysfunction in early-stage diabetes, and providing long-term protection from the development or onset of overt or insulin-dependent diabetes in individuals with impaired glucose tolerance. The invention also compares dihydrofolate activity with synthetic folate as well as the predominant biological folate derivative 5-methyltetrahydrofolate.

### FIELD OF THE INVENTION

The present invention relates to the novel use of dihydrofolate for its hypoglycemic activity. The nutraceutical use of dihydrofolate in managing diseases including type 1 and type 2 diabetes and associated complications is also provided. The invention is also directed to therapies for administering oral nutraceutical agent dihydrofolate on a chronic basis to individuals with pre-diabetes, including those with impaired glucose tolerance and/or insulin resistance, as well as early and late-stage diabetics. The invention also relates to the comparison of dihydrofolate against synthetic folate and 5-methyltetrahydrofolate.

### BACKGROUND OF THE INVENTION

Glucose is the central molecule of energy for mammals. Maintaining normal plasma glucose concentrations requires a balance between glucose utilization and endogenous glucose production or dietary glucose consumption, known as glucose homeostasis. Glucose homeostasis plays an important role in the human system as the required constant source of energy is mostly derived from the oxidation of glucose.

Abnormal blood glucose levels outside of the normal therapeutic range can have serious health consequences, including death, as the brain and red blood cells rely on glucose as their obligatory fuel source. This abnormal blood glucose level due to the abnormal metabolism of glucose leads to different metabolic disorders such as obesity and diabetes (type1 diabetes, type 2 diabetes, gestational diabetes)

The B group of vitamins is critical for glucose metabolism, and there is evidence that folate (Vitamin B9) consumption can improve glucose metabolism, reduce insulin resistance and decrease hepatic oxidative stress.

Folates are a group of vitamins that include synthetic folate and other biological derivatives which have been recognized as powerful antioxidants that protect cells from damage caused by oxidative stress.

Synthetic folate is biologically inactive and must be converted to its active forms in the body through a series of enzymatic reactions when consumed. However, the efficiency of this conversion in humans is very low.

Synthetic folate supplements are commonly used in many developed countries, and fortification of processed foods with folate is mandatory in some of these countries. However, there are concerns about using synthetic folate for fortification because it is not found in living organisms. Recently, health professionals have suggested using the biologically active derivative 5-methyltetrahydrofolate instead of synthetic folate for fortification purposes.

Parker, Samantha E., et al. American journal of obstetrics and gynecology 209.3 (2013): 239-e1. suggest that folate can reduce the risk of spina bifida associated with diabetes mellitus, though it may not eliminate it entirely.

Gargari, Bahram Pourghassem, Vahide Aghamohammadi, and Akbar Aliasgharzadeh, Diabetes research and clinical practice 94.1 (2011): 33-38, describe that folate supplementation has been shown to reduce the levels of homocysteine in the blood, improve glycemic control, and decrease insulin resistance in patients with type 2 diabetes.

Scambi, Cinzia, et al. PLOS One 4.3 (2009): e4782 describe that combined use of 5-methyltetrahydrofolate and vitamin B12 increases the bioavailability of active folate and reduces cellular injury caused by free radicals in children with cystic fibrosis.

Jacobs, Allen M., and Dunlei Cheng. Reviews in Neurological Diseases 8.1-2 (2011): 39-47, describes combination therapy using 5-methyltetrahydrofolate, methylcobalamin, and pyridoxal 5′-phosphate has been proposed as a treatment option for small-fiber neuropathy in patients with diabetes.

Walker Jr, Mackie J., Lauren M. Morris, and Dunlei Cheng. Reviews in Neurological Diseases 7.4 (2010): 132-139, describes that combined 5-methyltetrahydrofolate, methylcobalamin, and pyridoxal 5′-phosphate oral treatment has been shown to help restore lost cutaneous sensation in patients with diabetic peripheral neuropathy.

Wang, Jianhua, et al. Eye and vision6 (2019): 1-11. describe that the use of a specifically formulated medical food containing 5-methyltetrahydrofolate and vitamin D may aid in the improvement of diabetic retinopathy.

Asbaghi, Omid, et al. Nutrients 13.7 (2021): 2355, describe that a systematic review and dose-response meta-analysis of randomized controlled trials has shown that taking synthetic folate may improve blood sugar levels by reducing fasting blood glucose, and insulin resistance (as measured by HOMA-IR), without affecting long-term blood sugar control (as measured by HbA1c). However, these effects may not be substantial enough to have a significant impact on diabetes prevention and management.

Fohr, Iris P., et al. The American journal of clinical nutrition 75.2 (2002): 275-282, describe the treatment of 400 μg of folate with equimolar amounts of [6R,S]-5-methyltetrahydrofolate after 8 weeks of treatment, the serum folate concentrations were found to be significantly higher (˜3-fold) in the [6R,S]-5-methyltetrahydrofolate group compared to the folate group.

For the synthesis of tetrahydrofolate forms, first, the synthetic folate is converted to dihydrofolate, which is then reduced to tetrahydrofolate forms. Being an intermediatory molecule in the folate pathway, studies related to dihydrofolate is limited. No reports are available on the use of dihydrofolate for its hypoglycemic activity and use in the treatment of diabetes. The synthetic folate commercially used for fortification purposes is a structurally different molecule from dihydrofolate. Synthetic folate is having limited turnover rate in humans into biologically active folate derivatives. It is estimated that the efficiency to convert synthetic folate to biologically active derivatives in humans is less than 2% of that of rats. The studies available on the use of synthetic folate for the management of diabetes have shown limited activity. Nonetheless, the hypoglycemic activity of synthetic folate reported in the literature is limited.

### OBJECTIVES OF THE INVENTION

The main objective of the present invention is to provide the use of dihydrofolate as a glucose-lowering agent
One another objective of the present invention is to develop a novel antidiabetic nutraceutical for the management of diabetes (both type 1 and type 2) as well as associated complications.
The objective is also to use the current invention as a replacement for insulin in patients with type 2 diabetes, while still allowing for its use in combination with reduced insulin levels for those with type 1 diabetes.
Another objective of the present invention is to replace synthetic folate with the biological derivative dihydrofolate in the fortification program.
Yet another objective is to highlight the potential benefits of a compound that is not widely used or well-known or underutilized.

### SUMMARY OF THE INVENTION

The present invention pertains to the novel use of dihydrofolate as an active molecule for lowering blood glucose levels for its applications in managing glucose metabolism related disorders and associated complications which can be alleviated by control of blood sugar levels. The present invention can be used for treating hyperglycemia and related disorders in a mammal, by administration of a therapeutically effective amount of dihydrofolate to a mammal need thereof. The invention can be used to treat a variety of conditions and disorders such as dyslipidemia, cardiovascular disease, insulin resistance, obesity, and all types of diabetes including type 1, type 2, and gestational diabetes.

The present invention relates to the novel use of dihydrofolate as an active molecule for lowering blood glucose levels which presented significantly improved results compared to synthetic folate and 5-methyltetrahydrofolate.

The present invention relates to the use of the compound dihydrofolate as an active molecule for the management of patients with impaired glucose tolerance or early or late-stage diabetes. The compound provides positive and long-lasting therapeutic effects on the subject's glucose tolerance, glycemic control, and overall therapeutic effects that are greater than synthetic folate and 5-methyltetrahydrofolate.

The present invention is to provide a nutraceutical oral treatment of dihydrofolate for patients with impaired glucose tolerance or early or late-stage diabetes to improve the growth of and thereby function of pancreatic beta cells and the therapeutic effects that are greater than those of synthetic folate and 5-methyltetrahydrofolate.

The present invention is to provide nutraceutical oral treatment of dihydrofolate for patients with impaired glucose tolerance or early or late-stage diabetes without the negative side effects associated with traditional therapy which was better than synthetic folate and 5-methyltetrahydrofolate. This includes the avoidance of hypoglycemia as well as reducing the weight gain commonly seen with traditional approaches.

The present invention also aims to provide an effective nutraceutical for oral administration in the form of dihydrofolate as an effective agent for improving overall health, supporting specific bodily functions, particularly for the subject's diabetes, impaired glucose tolerance, and achieving glucose homeostasis, as well as early and late-stage diabetes with greater effect than synthetic folate and 5-methyltetrahydrofolate.

The present invention provides with composition comprising dihydrofolate for use in treatment of abnormal blood glucose levels in a subject. The composition can be in the form of a nutraceutical composition.

### BRIEF DESCRIPTION OF THE DRAWINGS

FIG. 1 shows a plot of the percent change of glucose values without any intervention for the addition of dihydrofolate, synthetic folate, and 5-methyltetrahydrofolate in rodent liver in in vitro assay.

FIG. 2 shows a plot of the percent change of glucose values without any intervention for the addition of dihydrofolate, synthetic folate, and 5-methyltetrahydrofolate in human serum in in vitro assay.

FIG. 3 explains the protective effect of dihydrofolate over synthetic folate and 5-methyltetrahydrofolate on high-glucose induced oxidative stress in in vitro assay.

FIG. 4 explains the protective effect of dihydrofolate over synthetic folate and 5-methyltetrahydrofolate on high glucose-induced inflammation in in vitro assay.

FIG. 5 explains a graph of the fasting blood glucose levels after administration of dihydrofolate, synthetic folate, and 5-methyltetrahydrofolate in diabetic rodents.

FIG. 6 illustrates a graph of the changes in whole blood glucose levels over a two hour interval after administration of glucose orally to rodents administrated separately with dihydrofolate, synthetic folate, 5-methyltetrahydrofolate in diabetic rodents.

FIG. 7 explains organ histology after administration of dihydrofolate, synthetic folate, and 5-methyltetrahydrofolate in diabetic rodents.

### DETAIL DESCRIPTION OF THE INVENTION

The present invention provides a method of treating hyperglycemia and related disorder in a mammal, the method comprising administering a therapeutically effective amount of dihydrofolate to the mammal.

In an embodiment, the related disorder is selected from a group consisting of dyslipidemia, cardiovascular diseases, insulin resistance, obesity, diabetes. Diabetes can either be type 1 diabetes, type 2 diabetes or gestational diabetes.

In a preferred embodiment, a therapeutically effective amount of dihydrofolate is administered orally or through oral route to the mammal, preferably a human being.

Preferably dosage form of this invention is to be taken for at least one day, and more preferably on a chronic basis, for the duration of the patient's disease or disorder management.

In a preferred embodiment, the nutraceutical agent dihydrofolate or a nutraceutical composition comprising dihydrofolate can be administered in multiple doses daily, ideally at bedtime and before each meal, specifically before breakfast, lunch, and dinner. It is even more preferable to administer the compound at or shortly before bedtime and within 15 minutes or less of consuming a meal. The dihydrofolate can also be administered along with the meal.

Preferably the therapeutic agent dihydrofolate will be orally administrated to patients with impaired glucose tolerance, such as those with HbA1c levels ranging from normal to elevated. This includes patients with insulin resistance in the pre-diabetic and early stages of Type 2 diabetes, as well as those with failure of insulin production in Type 1 diabetes and advanced stages of Type 2 diabetes. Specifically, the nutraceutical can be administered to individuals with varying levels of glycemic control, including normal, impaired, and late-stage Type 2 or Type 1 diabetes.

In preferred embodiments, the mammal's glucose tolerance is improved after dihydrofolate administration, as shown by:
Mammal's ability to metabolize sugar is improved, as evidenced by a statistically significant decrease in fasting blood glucose levels compared to baseline levels before treatment with better effect than synthetic folate 5-methyltetrahydrofolate.
In certain preferred embodiments, the mammal's ability to regulate glucose levels is improved compared to non-treatment mammals, without a statistically significant increase in weight or risk of low or high blood sugar levels over the treatment period.
In preferred embodiments, the oral dosage form is solid and is preferably delivered in a gelatin capsule or tablet.
In certain preferred embodiments, the oral absorption of dihydrofolate can be confirmed by observing the decrease in blood glucose levels in a subject after the oral administration of the compound. The decrease in blood glucose levels will vary depending on the dose of dihydrofolate. In these preferred embodiments, the compound is designed to provide maximum control of glucose levels at 0.5-1.5 hours, more preferably at 0.75 to 1 hour, after oral administration. Additionally, the composition is designed to provide maximum post-prandial glucose control at less than 120 minutes, more preferably at less than 80 minutes, and still more preferably at 45 to 60 minutes, after oral administration.
In certain preferred embodiments, the dosage form(s) of the nutraceutical agent between 1-200 mg/kg body weight of the compound, with 100-150 mg being preferred, 150-100 mg/kg body weight being more preferred, 100-50 mg/kg body weight being even more preferred, and 50, 250, or 10 mg/kg body weight is the most preferred.
For purposes of the present invention, the antidiabetic agent administered orally is a hypoglycemic agent, specifically vitamin folate derivative. In preferred embodiments of the invention, dihydrofolate is anticipated, that will facilitate the attainment of normoglycemia, will result in a reduction in HbA1C levels within the desired goal, spare pancreatic beta cells, delay or prevent the progression of diabetes, and defer or eliminate the need to resort to injectable insulin or other medications. An oral administration of dihydrofolate is likely to offer great advantages in the management of patients with type 2 diabetes.

The definitions for the following terms will be used throughout the application:

Patient—is any mammal that has been determined to have a condition requiring medical attention or treatment.

Subject—refers to a person or group of people who are being studied or experimented on.

Diabetic patient—refers to a mammal that has been diagnosed or suspected of having pre-diabetes or diabetes, and/or has a condition that would benefit from treatment with anti-diabetic medications and/or insulin therapy.

Mammal—a type of animal that encompasses various species, including but not limited to: rodents, aquatic mammals, domestic pets such as dogs and cats, and farm animals such as sheep, pigs, cows, and horses, with a focus desirably on humans or human beings.

Diabetes or Diabetes Mellitus—refers to both Type 1 and Type 2 diabetes, unless otherwise stated specifically.

Early-stage diabetes—refers to a condition where there is impaired control of blood sugar levels, without treatment where in the pancreas islet cells are still functioning, but in a compromised state. This includes conditions such as impaired glucose tolerance and impaired fasting blood glucose, where the body's own insulin production is not enough to fully control blood sugar levels after eating, but is still able to provide some control.

Late-stage diabetes—refers to a condition where there is impaired control of blood sugar levels, without treatment. In this stage, the pancreas' islet cells are either failing or have failed completely. This means that the body's own insulin production is no longer able to control blood sugar levels after eating, either in the short or long term.

Treatment—as used in this context includes not only managing the symptoms of diabetes but also preventing the onset of diabetes, delaying the progression of the condition, and slowing the worsening of diabetic conditions unless specifically stated otherwise.

Nutraceutical agent—the active molecule/compound/drug/nutraceutical/medication disclosed by reference herein is a chemical substance that is used to diagnose, cure, treat, or prevent a disease or medical condition. The nutraceutical agent in accordance with the present invention is dihydrofolate.

Nutraceutical composition—refers to a composition comprising dihydrofolate in a therapeutically effective amount. The composition may additionally comprise pharmaceutically acceptable excipients, carriers, and/or diluents. Preparation of such a composition is within the ability of a skilled person.

Compound—refers in this context to a specific chemical compound that is being studied or researched for a particular purpose.

Molecule—refers to a specific chemical molecule that is being studied or researched herein.

An effective dose or therapeutically effective amount of a nutraceutical agent—refers to the amount of the active molecule or its derivatives, including salts, solvates, active metabolites, prodrugs, or enantiomers, that when given to a mammal, is enough to treat or prevent a disease, state or condition. The amount will vary based on factors such as the severity of the condition being treated, the age, weight, and overall health of the mammal being treated.

Dosage form—refers to a discrete unit that is suitable for human or animal subjects and is packaged individually as is known in the art. The invention contemplates that dosage forms may include one or more unit doses such as tablets, capsules, powders, semisolids (e.g. gelcaps or films), liquids for oral administration, ampoules or vials for injection, or loaded syringes to achieve the therapeutic effect. A preferred embodiment of the dosage form is an oral dosage form.

Multiple doses—refers to a situation where a patient has received at least two doses of a drug/nutraceutical within the dosing interval established for that specific drug/nutraceutical.

Single dose—refer to a situation where a patient has received a single administration of a drug/nutraceutical composition or when repeated single doses have been given with washout periods in between.

Unless otherwise specified as “single dose” or “steady-state”, the pharmacokinetic parameters disclosed and claimed in this context, refer to both single-dose and multiple-dose conditions.

Meal as used herein refers to a standard diet

The term “AUC” as used in this context refers to the area calculated under the curve of the plasma concentration over time.

The phrase “night time” or “bedtime” as mentioned in this context refers to the time before a patient falls asleep and is not restricted to a specific time of day or light/dark cycle, and may also refer to a period during the day or night when the patient is fasting for the longest duration, without consuming any external sources of glucose.

The phrase “administered at night time or at or shortly before bedtime” as used in this specification means that the medication should be given less than 3 hours, preferably less than 2 hours, and more preferably less than 1 hour before a prolonged period of sleep or inactivity and fasting, such as overnight. This period typically refers to the late night to early morning hours, but can also refer to any period of a sleep-wake cycle during which a person obtains their necessary sleep. Additionally, the medication should be given at least 1 hour, preferably 1.5 hours, more preferably 2 hours, and still more preferably 2-3 hours after the last meal of the day.

The singular forms of “a,” “an,” and “the” in this document and in the appended claims refer to multiple items unless the context indicates otherwise. For example, “a molecule” can refer to one or more molecules, “a reagent” can refer to one or more different reagents and “the method” can include equivalent steps and methods known to those in the field that can be substituted for the methods described.

As used in this context, the term “treat” refers to one or more of the following actions:
Manage or attempt to improve a medical condition or disease.
Preventing the onset or worsening of a condition, managing symptoms, and promoting healing or recovery.
Preventing, curing, healing, alleviating, relieving, altering, remedying, improving, or affecting a condition such as a disease, the symptoms of the condition, or the predisposition towards the condition.

Unless otherwise defined, all technical and scientific terms used in this embodiment have the same meaning as commonly understood by an expert in the field to which this invention belongs. Although other methods, compositions, reagents, cells, or similar may be used in the practice or testing of the invention, the preferred methods and materials are described in this document. All publications mentioned in this document are incorporated by reference, including figures, graphs, equations, illustrations, and drawings, to provide specific information for which the reference was cited.

The publications discussed above are included for the purpose of disclosing information prior to the filing date of this application. It should not be interpreted as an admission that the invention is not entitled to be considered as prior art. The preferred embodiment and examples shown throughout this description should be regarded as examples rather than as limitations on the present invention.

The regular use of the oral medication described in this invention is thought to provide better control of blood sugar levels and improved glucose tolerance compared to before treatment, without causing any significant changes in weight or increasing the risk of hypoglycemia during the treatment period. Additionally, it is believed that the regular use of this medication leads to better utilization, and secretion of insulin, and improved HbA1C levels.

It is also believed that using the oral medication described in this invention on a regular basis, can replace the natural insulin production in mammals with impaired glucose tolerance, which will help to preserve the function of the mammal's beta cells or prevent their death or dysfunction, and thus provide long-term protection from developing severe diabetes, or delay the onset of these conditions in the mammal.

The preferred form of the medication described in this invention is a single molecule of dihydrofolate. This medication can be delivered in various forms such as a capsule, tablet, solution, freeze-dried powder, or suspension, ready for administration to a patient. The preferred form being of a gelatin capsule or tablet.

The term “dihydrofolate” used herein is the natural form of vitamin folate, which is generally produced as an intermediary molecule in the folate metabolism pathway.

The appropriate amount of dihydrofolate to be used can be determined by experts in the field. It is ideal for the oral form of the medication to contain a sufficient amount of dihydrofolate that is effective in treating the condition or achieving the desired results. The recommended dosage of dihydrofolate should be such that it leads to a noticeable and statistically significant decrease in blood sugar levels in healthy human test subjects when taken orally. The appropriate amount of dihydrofolate can be therapeutically effective amount of dihydrofolate.

The preferred doses of dihydrofolate described, range from 50 to 600 mg with optimal doses being between 300 and 450 mg or even more specifically between 200 and 300 mg with the most preferred dose being 100 mg considering the conversion factor of rodents and humans.

It is worth noting that the appropriate dose of dihydrofolate may vary depending on the individual and the severity of the condition being treated. Additionally, it may result in synergistic effects when taken as a nutraceutical.

The oral dosage forms described in this invention are designed to lower the risk of hypoglycemic events. The oral forms of the drug administration is more practical and safer for patients, as it allows the drug to be taken shortly before a meal and thus glucose is ingested shortly after. Its water-soluble nature allows for efficient absorption and utilization of the drug.

The compound discussed in this embodiment can be created from synthetic folate and can be made using methods familiar to those with expertise in the field, as outlined in the current information provided and the methods described in international publications.

The absorption of dihydrofolate can be measured by monitoring the levels of the molecule in the bloodstream after treatment. The time it takes for the dihydrofolate to reach its peak in the bloodstream can vary depending on factors such as the type of dosage form (solid, liquid, tablet, capsule, and suspension), the concentration of the molecule and the delivery agent in the gastrointestinal tract, the subject's feeding state, diet, and overall health.

Dihydrofolate does help patients who have insulin-dependent or type 1 diabetes as its action can be independent of insulin. It can also act in the absence of insulin however the tissues which solely depend on insulin for the uptake of glucose require some amount of insulin in body. In such cases, dihydrofolate can be administrated along with insulin.

After taking the dosage, the dihydrofolate is absorbed into the bloodstream and the levels of dihydrofolate in the blood can be measured. The effectiveness of dihydrofolate can be evaluated by measuring a specific biological effect in the blood, such as a reduction in blood glucose levels. Additional physiological effects of the compound can also be evaluated by conducting tests, for example, by measuring the concentration of C-peptide in the plasma to determine the level of insulin produced by the body thereby identifying the improvement in pancreatic beta cell function or measuring HbA1c levels in long run.

In addition, a lipid profile test can be performed to gauge a diabetic patient's glucose control over the previous two to three weeks. A lipid profile is a blood test that measures the levels of triglycerides and cholesterol in the blood. Elevated levels of this LDL cholesterol and triglycerides are associated with an increased risk of diabetes. While blood glucose levels provide a short-term indication of diabetes control, a lipid profile test is a short- to a medium-term marker that can indicate the patient's overall cardiovascular health and the effectiveness of the diabetes treatment over a two-week period.

Improved glycemic control can be achieved by observing:
A reduction in fasting blood glucose levels by measuring glucose levels that are statistically lower than the baseline levels before treatment.
Postprandial blood glucose concentration that is reduced by a statistically significant amount as compared to the baseline postprandial blood glucose concentration prior to treatment.
The improved glycemic control can be demonstrated by a statistically significant decrease in HbA1c levels
An improvement in glucose tolerance test results in a reduction in the area under the curve (AUC) of blood glucose excursion following a glucose load, compared to the AUC prior to treatment.
A reduction in the need for exogenous insulin or other diabetes medication. A decrease in the dosage or frequency of insulin or other diabetes medication is required to maintain glycemic control as compared to baseline levels prior to supplementation. This can be tracked by monitoring the patient's glucose levels and medication regimen over time.

It is preferred that dihydrofolate is administrated to human patients on a chronic basis, even for the duration of the patient's life, with the option of alternating on and off periods.

It is believed that chronic administration of the oral dosage forms of the invention will decrease the rate and intensity of systemic hyperinsulinemia caused by chronic dosing of insulin or by certain conditions associated with chronic dosing of insulin in a mammal with impaired glucose tolerance or early-stage diabetes.

The invention offers a way to decrease the occurrence and/or intensity of high insulin/other medications in the body caused by prolonged use, and it is believed that it also reduces the likelihood and/or intensity of various health issues caused by prolonged use of insulin/other medication as the present invention relates to a vitamin derivative.

The administration of the oral nutraceutical compound should be tailored according to the patient's specific needs for glycemic control. For example, if the patient needs to control fasting glucose levels, the formulation should be taken at or shortly before bedtime. If the patient needs to control post-prandial glucose levels, it should be taken before some meals. If the patient needs total post-prandial control, the formulation should be taken before all meals. For comprehensive glycemic control, the compound should be taken before all meals and at or shortly before bedtime.

It is still further believed that the present invention provides a method of chronic administration of oral dosage forms that can replace endogenous insulin production in mammals with impaired glucose tolerance or early-stage diabetes mellitus. This aims to prophylactically preserve the function of the mammal's beta cells and prevent death or dysfunction of the mammal's beta cells, thereby providing long-term protection against the development of overt or insulin-dependent diabetes or delaying its onset.

The following examples are provided to illustrate the invention and should not be interpreted as limiting the scope of the invention in any way.

### EXAMPLES

Following examples are given by way of illustration and therefore should not be construed to limit the scope of the invention.

### Example-1

The Study Aimed to Assess the Effectiveness of Dihydrofolate Over Synthetic Folate and 5-Methyltetrahydrofolate by In Vitro Glucose Assay Using Rat Liver Homogenate.

Rat liver homogenate was treated with 100 μM glucose in the absence or presence of 25 μM or 50 μM dihydrofolate/synthetic folate/5-methyltetrahydrofolate. The glucose concentration was determined after incubation at 37° C. for 1 hour.

FIG. 1. shows the effect of folates on the percentage reduction in glucose content on glucose treated liver homogenate. (A) Folates at a concentration of 25 μM. (B) Folates at 50 μM. Control represents glucose concentration without folate treatment which is considered to be 100%. The results are presented as mean±SD (n=3), and the significance of the values was analyzed by one-way ANOVA followed by Tukey's test (p<0.05). *p<0.05, **p<0.01, *p<0.001, ***p<0.0001. FA: synthetic folate, MTHF: 5-methyl tetrahydrofolate, DHF: dihydrofolate.

As can be seen, glucose concentration decreased upon dihydrofolate and 5-methyltetrahydrofolate treatment with no effect on synthetic folate treatment. The greater glucose lowering was shown by dihydrofolate.

### Example 2

Human serum was treated with 100 μM glucose in the absence or presence of 50, 100, 200, 400, or 600 μM dihydrofolate/synthetic folate/5-methyltetrahydrofolate. The glucose concentration was determined after incubation at 37° C. for 1 hour.

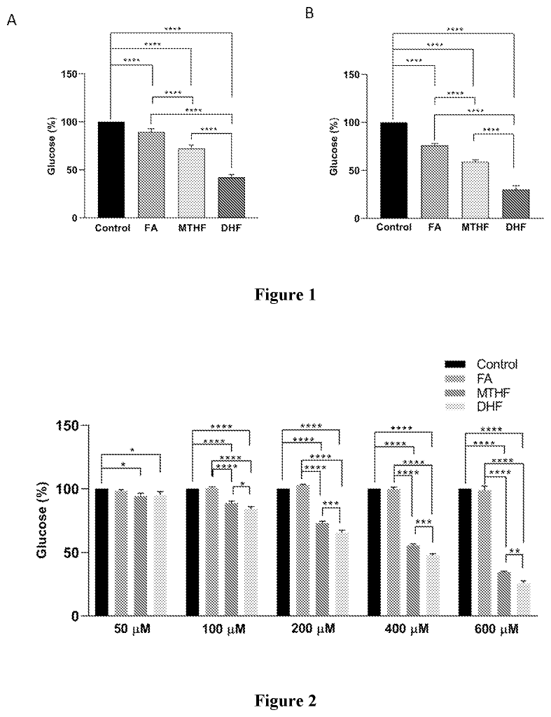

FIG. 2. shows the effect of folates (50, 100, 200, 400, 600 μM) on the percentage reduction in glucose content in glucose-treated human serum. Control represent glucose concentration without folates treatment which is considered to be 100%. The results are presented as mean±SD (n=3), and the significance of the values was analysed by one-way ANOVA followed by Tukey's test (p<0.05). *p<0.05, **p<0.01, ***p<0.001, ****p<0.0001 except for 50 μM where (p<0.02). *p<0.02. FA: synthetic folate, MTHF: 5-methyl tetrahydrofolate, DHF: dihydrofolate.

### Example 3

Protective Effect of Folates from High Glucose-Induced Oxidative Stress

Human bronchial epithelial cells were co-treated with high glucose (50 μM) and 0.012 g/L dihydrofolate/synthetic folate/5-methyltetrahydrofolate. Cell viability, reactive oxygen species production using DCFH-DA (in terms of microtitre plate assay was determined as well as the microscopic examination was carried out.

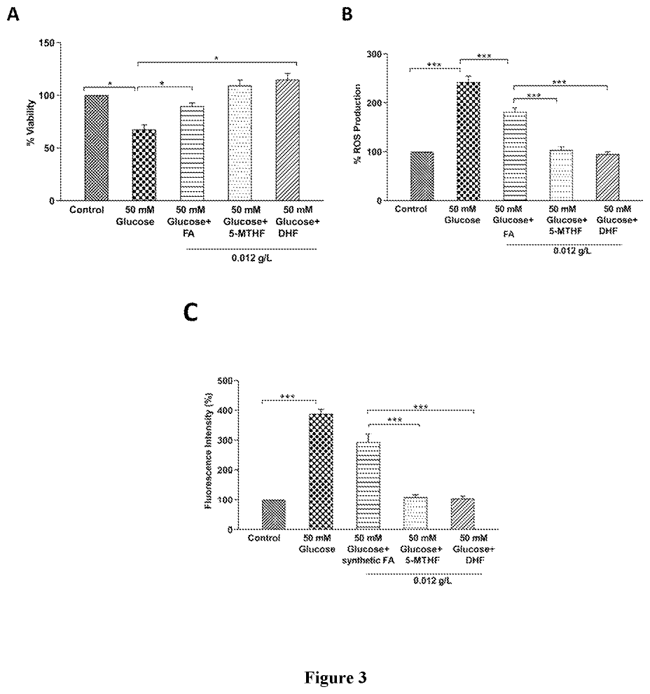

FIG. 3. shows cell viability and ROS production in BEAS-2B cells grown in high glucose medium and supplemented with 0.012 g/L of synthetic folate or folate derivatives 5-methyltetrahydrofolate and dihydrofolate (A) Percentage cell viability (B) Percentage ROS production (C) Quantification of ROS production in terms of fluorescence intensity of microscopic images by ImageJ software. The results are presented as mean±SD (n=3), and the significance of the values was analyzed by one-way ANOVA followed by Tukey's test (p<0.05). *p<0.05, **p<0.01, ***p<0.001. FA: synthetic folate, MTHF: 5-methyl tetrahydrofolate, DHF: dihydrofolate.

### Example 4

Protective Effect of Folates from High Glucose-Induced Inflammation

Example 3 was repeated but instead of oxidative stress marker inflammatory marker production was analyzed. NfkB protein estimation using western blot analysis of cell lysate homogenate. Other inflammatory markers IL-6, TNFα, and MCP-1 were measured by measuring the concentration of these markers in the cell culture supernatants using the ELIZA kit method.

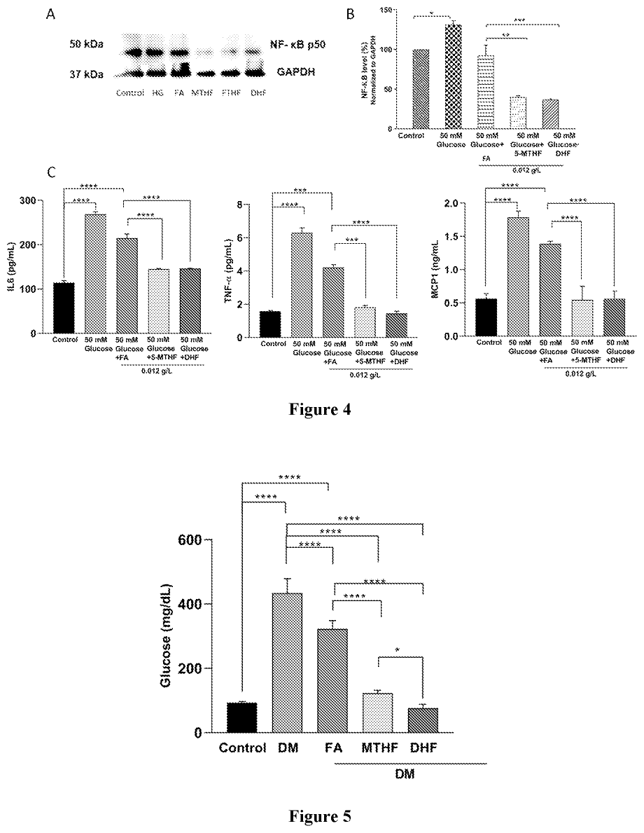

FIG. 4. depicts protective effect of folates on high glucose-induced inflammation in BEAS-2B cells. (A) Western blot analysis of NF-κB p50 protein expression (B) Quantification of NF-κB p50 protein bands normalized to GAPDH using densitometric analysis by Image Lab software (C) Inflammatory cytokine and chemokine production in cell culture supernatants. The results are presented as mean±SD (n=3), and the significance of the values was analyzed by one-way ANOVA followed by Tukey's test (p<0.05). *p<0.05, **p<0.01, ***p<0.001. FA: synthetic folate, MTHF: 5-methyl tetrahydrofolate, DHF: dihydrofolate, FTHF; 10-formyl tetrahydrofolate.

### Example 5

This example describes the results of a study wherein dihydrofolate/synthetic folate/5-methyltetrahydrofolate was administered to diabetic Wistar rats in order to determine the efficacy of the compound. Dosing solutions s were prepared by dissolving dihydrofolate/synthetic folate/5-methyltetrahydrofolate in water. Final dosing solutions contained 5 mg/kg body weight either dihydrofolate or synthetic folate or 5-methyltetrahydrofolate. Fasting blood sugar concentration was estimated.

FIG. 5. Effect of folates (5 mg/kg body weight folate equivalent) on fasting blood glucose level in diabetic Wistar rats. The results are presented as mean±SD (n=6), and the significance of the values was analyzed by one-way ANOVA followed by Tukey's test (p<0.05). *p<0.05, **p<0.01, ***p<0.001, ****p<0.0001. DM: diabetic group, FA: synthetic folate, MTHF: 5-methyl tetrahydrofolate, DHF: dihydrofolate.

### Example 6

### Oral Glucose Tolerance Test

Oral glucose tolerance test (OGTT). The test was performed by oral administration of glucose (2 g/kg body wt). Graph showing blood glucose concentration before (0 min) and 30, 60, 90, and 120 min after administration. Data are shown as the mean±SEM (n=6).

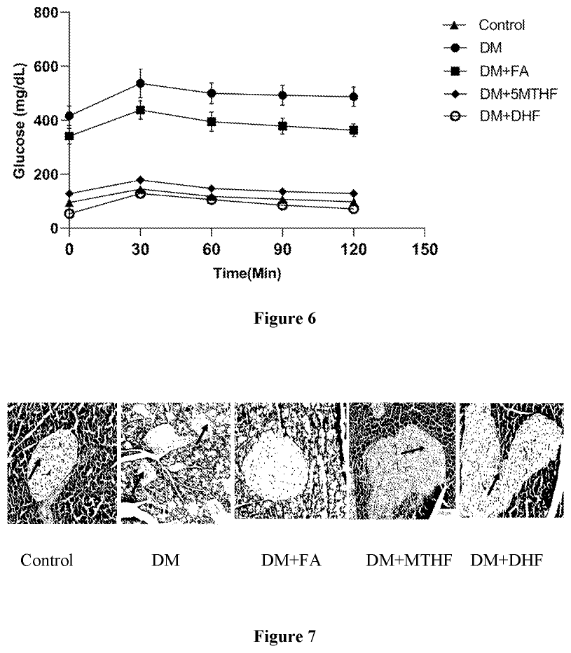

As can be seen in FIG. 6, diabetic rats show a significantly improved glucose tolerance upon compound administration with greater effect for dihydrofolate.

The rats receiving 5-methyltetrahydrofolate experienced a gradual decline in whole blood glucose values from baseline, which then leveled off after about 120 minutes, also lasting into the end of the testing period. The rats administrated with dihydrofolate had a more gradual decline in whole blood glucose than the 5-methyltetrahydrofolate that lasted until the end of the study. The rats administrated with synthetic folate did not show much declaim in the blood glucose level.

The oral administration of the folate derivatives tested in this study showed a rapid onset of hypoglycemic action within 30 minutes of oral administration with a greater effect by dihydrofolate over synthetic folate and 5-methyltetrahydrofolate. The hypoglycemic effect lasted for at least 4 hours when the testing was stopped. However, it is believed that the glycemic control may continue beyond the time tested. The composition also provided a decrease in the whole blood glucose concentration, starting around 20 minutes after oral administration, with the maximum decrease occurring around 180 minutes after oral administration. This glucose-lowering effect lasted for at least 5 hours after the initial dose.

### Example 7

Table 1. shows experiments with the diabetic Wistar rats. Long-term oral administration with folates (dihydrofolate/synthetic folate/5-methyltetrahydrofolic) significantly improved organ weight without inducing obesity. The results are presented as mean±SD (n=6), and the significance of the values was analyzed by one-way ANOVA followed by Tukey's test (p<0.05). *p<0.05, **p<0.01, ***p<0.001, ****p<0.0001. DM: diabetic group, FA: synthetic folate, MTHF: 5-methyl tetrahydrofolate, DHF: dihydrofolate.

TABLE 1
Organ weight

| | Control | DM | DM + FA | DM + MTHF | DM + DHF |
|---|---|---|---|---|---|
| Adipose | 9.89 ± 2.11 | 0.53 ± 0.43**** | 0.87 ± 0.60**** | 2.77 ± 2.11** | 5.43 ± 1.75* |
| Brain | 1.69 ± 0.12 | 1.30 ± 0.18**** | 1.33 ± 0.23*** | 1.46 ± 0.18 | 1.64 ± .09 |
| Cecum | 3.18 ± 0.66 | 6.16 ± 2.42 | 6.46 ± 1.49 | 4.33 ± 0.95 | 2.72 ± 0.51 |
| matter | | | | | |
| Heart | 0.95 ± 0.17 | 0.69 ± 0.14 | 0.68 ± 0.07 | 0.82 ± 0.23 | 0.88 ± 0.07 |
| Kidney | 2.06 ± 0.23 | 1.98 ± 0.27 | 2.12 ± 0.46 | 2.12 ± 0.2 | 1.74 ± 0.21 |
| Liver | 11.75 ± 1.83 | 6.86 ± 1.17** | 7.35 ± 1.02 | 8.49 ± 2.52 | 10.68 ± 1.46 |
| Lungs | 2.24 ± 0.31 | 1.58 ± 0.28 | 1.46 ± 0.38 | 1.93 ± 0.62 | 2.09 ± 0.18 |
| Spleen | 1.12 ± 0.12 | 0.63 ± 0.21* | 0.66 ± 0.08 | 0.92 ± 0.35 | 1.09 ± 0.10 |
| Testes | 3.57 ± 0.86 | 1.79 ± 0.73* | 2.57 ± 0.95 | 2.31 ± 0.79 | 2.88 ± 0.27 |

### Example 7

FIG. 7 shows experiments with diabetic Wistar rats. Long-term oral administration with folates (dihydrofolate/synthetic folate/5-methyltetrahydrofolic) significantly improved islets of the pancreas with the beta cell area significantly larger compared to the non-treated diabetic group. The greater effect was shown by dihydrofolate compared to synthetic folate and 5-methyltetrahydrofolate. Therefore, long-term treatment with dihydrofolate is beneficial for beta cell function and blood glucose control rat model for type 1 diabetes.
Control—Normal morphology of beta cells of islets of the pancreas
Diabetic—Moderate atrophy of islets of the pancreas with apoptosis of beta cells was observed
Folate treatment group—Moderate atrophy of islets of the pancreas with apoptosis of beta cells was observed
5-methyltetrahydrofolate treated group—Moderate hypertrophy of islets of the pancreas and moderate hyperplasia of beta cells
Dihydrofolate treated group—Severe hypertrophy of islets of the pancreas with hyperplasia of beta cells were observed; positive response of the treatment

We claim:

1. A method of treating hyperglycemia and related disorder in a mammal, the method comprising administering a therapeutically effective amount of dihydrofolate to the mammal.

2. The method as claimed in claim 1, wherein the related disorder is selected from a group consisting of dyslipidemia, cardiovascular diseases, insulin resistance, obesity, diabetes.

3. The method as claimed in claim 2, wherein the diabetes is selected from type 1 diabetes, type 2 diabetes or gestational diabetes.

4. The method as claimed in claim 1, wherein the administering is preferably by oral route.

5. The method as claimed in claim 1, wherein the administering is done before meal, after meal or along with the meal.

6. The method as claimed in claim 1, wherein the therapeutically effective amount of dihydrofolate is in a range of about 1-200 mg/kg body weight, preferably 100-150 mg, more preferably 150-100 mg/kg body weight or 100-50 mg/kg body weight, and most preferably 50, 250, or 10 mg/kg body weight of the mammal.

7. The method as claimed in claim 1, wherein dihydrofolate is in the form of a nutraceutical agent.

8. The method as claimed in claim 1, wherein the mammal is a human being with diabetes.

9. The method as claimed in claim 8, wherein the diabetes is characterized by prediabetes or early stages of diabetes, or late stages of diabetes.

10. The method as claimed in claim 8, wherein the diabetes can be non-insulin dependent diabetes or insulin-dependent diabetes.

11. A composition comprising dihydrofolate for use in treatment of abnormal blood glucose levels in a subject.

12. The composition as claimed in claim 11, wherein the composition is an antidiabetic nutraceutical composition.

13. The composition as claimed in claim 12, wherein the composition is in the form of a gelatin capsule or tablet.
